(19) United States
(12) Patent Application Publication
(10) Pub. No.: US 2015/0159224 A1
(43) Pub. Date: Jun. 11, 2015

(54) ULTRASENSITIVE DETECTION AND CHARACTERIZATION OF CLUSTERED KRAS MUTATIONS USING PEPTIDE NUCLEIC ACID CLAMP PCR IN DROP-BASED MICROFLUIDICS

(71) Applicants: Huidan ZHANG, Cambridge, MA (US); Ralph SPERLING, Cambridge, MA (US); Neil DAVEY, Gaithersburg, MD (US); David A. WEITZ, Cambridge, MA (US)

(72) Inventors: Huidan ZHANG, Cambridge, MA (US); Ralph SPERLING, Cambridge, MA (US); Neil DAVEY, Gaithersburg, MD (US); David A. WEITZ, Cambridge, MA (US)

(21) Appl. No.: 14/541,126

(22) Filed: Nov. 13, 2014

Related U.S. Application Data
(60) Provisional application No. 61/903,857, filed on Nov. 13, 2013.

(51) Int. Cl. C12Q 1/68

(52) U.S. Cl. CPC C12Q 1/6886; C12Q 2600/156; C12Q 2600/106

(57) ABSTRACT

This disclosure employs the combination of a microfluidics platform and drop-based digital polymerase chain reaction (dPCR) to create a breakthrough technology that enables the detection of CTC genes and the isolation of single CTCs from the blood. In the first method, cDNA molecules from lysed CTCs are amplified in microfluidic drops and detected via fluorescence signal. In the second method, intact single CTCs are encapsulated, and amplification-positive drops are sorted from the remaining cells. To demonstrate the clinical utility of our technology, mutations in the KRAS gene in colorectal cancer are analyzed to study resistance to EGFR-based treatment as a test case. The methods herein present robust techniques for both the diagnosis and treatment of cancers, as well as for the obtainment of a pure CTC sample from billions of other cells in the blood.

### CROSS REFERENCE TO RELATED APPLICATION(S)

This application claims the benefit of U.S. Provisional Application No. 61/903,857 filed on Nov. 13, 2013, the content of which is incorporated herein in its entirety by reference.

### FIELD OF THE INVENTION

This invention is directed to cancer diagnosis using drop-based microfluidics.

### BACKGROUND

Cancer is a leading cause of death worldwide, and has been a pressing concern on the forefront of medical research for decades. The American Cancer society estimates 1,660,290 new cancer cases and 580,350 cancer-related deaths in 2013 in the United States alone. The cancers that have the greatest mortality rates in the United States include prostate, lung, breast, and colorectal. Lack of early cancer detection methodologies has resulted in low survival rates of cancer patients. Traditional diagnosis involves tumor biopsy, a technique that is highly invasive, dangerous, and often arbitrary; a doctor cannot be certain of the anatomical coordinates of a tumor and must poke a needle around an organ many times before accurately detecting the tumor site. Thus, there is an acute demand to perform early-stage non-invasive liquid biopsies, in which tumor cells are detected directly from a blood sample. Liquid biopsy would allow easier diagnosis of cancers and the ability to monitor cancer prognosis.

Circulating tumor cells (CTCs) are shed from a primary tumor into the vasculature and subsequently circulate in the bloodstream through a process known as metastasis. The seeding of CTCs, the byproducts of the primary tumor, to create secondary tumors triggers a mechanism that is responsible for the vast majority of cancer-related deaths. Thus, detecting CTCs at an early stage of cancer is of great importance since CTCs contain genetic abnormalities of cells within the original tumor masses and can reveal information about the progression of the cancer. Further, screening for genetic abnormalities in CTCs from the blood would enable oncologists to prevent dissemination of primary tumors and determine the drug therapy most effective in attacking a specific tumor type, such as EGFR-targeted therapies in colorectal cancer based on the presence of mutations in the KRAS gene. However, detecting CTCs from the bloodstream is a highly challenging task. Previous estimates showed that per milliliter of whole blood, there are only 1-10 CTCs among >1 billion red blood cells (RBCs) and >1 million white blood cells (WBCs). In addition to their extreme rarity, CTCs are highly heterogeneous, and no universal marker exists to identify CTCs originating from various cancers.

Current methods for the detection and isolation of CTCs, which are between 10-20 μm in diameter, include techniques based on size (centrifugation, microfilters, hydrodynamic sorting), immunocapture (micromixers, micropillar arrays, magnetic microbeads) and microscopy (non-porous glass or porous polymer substrates). However, none of these methods present a high-throughput platform that is both specific in ensuring that the final product contains only pure CTCs and sensitive in capturing all CTCs that were present the initial sample. Size-based devices capture a wide variety of unwanted cells (such as leukocytes), immunocapture fails to capture the full heterogeneous CTC population that was originally among billions of other cells in the blood sample, and microscopic examination of thousands of stained cells is extremely tedious and requires the cancer cells to be fixed.

The most state-of-the-art CTC isolation technology, known as the CTC Inertial Focusing Chip (iChip) (FIG. 1), combines these three techniques to decrease time and increase sensitivity and specificity. Size-selection is used to deplete RBCs and immunoaffinity-based magnetic bead-selection is used to deplete WBCs from a whole blood sample in an attempt to purify CTCs. With this technology, a 10 mL blood sample can be concentrated to a 100 μL product containing about 500,000 RBCs, about 5,000 WBCs, and an unknown number of CTCs within one hour. Despite these advancements, detection and isolation of CTCs from a mixture of about 505,000 cells employing a high throughput method still remains an unresolved challenge. Subsequently, CTC detection is accomplished by microscopic examination of thousands of cells stained with antibodies to surface markers associated with CTCs, a technique that is time consuming and often error prone.

### SUMMARY

Disclosed herein is a method for diagnosing cancer in a person, comprising: obtaining or preparing a sample of the person, the sample comprising cDNAs of a plurality of genes of the person; encapsulate the cDNAs into discrete droplets, wherein statistically each of the discrete droplets contains at most one of the cDNAs; amplifying the cDNAs in the droplets; determining whether the droplets contain a cDNA of a mutation of a V-Ki-ras2 Kirsten rat sarcoma viral oncogene homolog (KRAS) gene.

Disclosed herein is a method for diagnosing cancer in a person, comprising: obtaining or preparing a sample of the person, the sample comprising whole cells of the person; encapsulate the whole cells into discrete droplets, wherein statistically each of the discrete droplets contains at most one of the whole cell; lysing the whole cells in the droplets; forming cDNAs by reverse transcribing mRNAs in lysate in the droplets; amplifying cDNAs in the droplets; determining whether the droplets contain a cDNA of a mutation of a KRAS gene.

According to an embodiment, the method further comprises sorting the droplets.

According to an embodiment, the sample is a whole blood sample.

According to an embodiment, obtaining the sample comprises reverse transcribing mRNAs.

According to an embodiment, the cancer is colorectal cancer.

According to an embodiment, the cancer is prostate cancer.

According to an embodiment, the mutation is codon 12 or codon 13 of the KRAS gene.

According to an embodiment, the mutation is alteration of a guanine in the KRAS gene.

According to an embodiment, determining whether the droplets contain a cDNA of a mutation of the KRAS gene is by using peptide nucleic acid (PNA) clamping.

According to an embodiment, determining whether the droplets contain a cDNA of a mutation of the KRAS gene is by using a fluorescence indicator.

According to an embodiment, the person is suspected of having cancer.

According to an embodiment, the method further comprises determining the sequence of the mutation.

According to an embodiment, the method further comprises selecting a therapy for the person based on the sequence of the mutation.

According to an embodiment, the therapy comprising introducing an antibody into the person.

### BRIEF DESCRIPTION OF THE DRAWINGS

FIG. 1: CTC iChip (image obtained from). 10 mL of whole blood is inputted to the chip. Via size-selection, RBCs and platelets are depleted from the blood. WBCs are then depleted via magnetic bead-selection, resulting in a 100 μL product that contains about 500,000 RBCs, about 5,000 WBCs, and an unknown number of CTCs.

FIG. 2a: Workflow for cDNA dPCR. Cells were pooled together and lysed, and their mRNA was subsequently extracted. Following reverse transcription (RT), cDNA molecules were encapsulated to make 20 μm drops, in which PCR amplification was performed. Fluorescence of the drops with positive amplification was finally detected at laser point using a microfluidic-based flow cytometer.

FIG. 2b: Encapsulation step for single-cell dPCR. Whole single cells were co-encapsulated with the PCR mix and lysis buffer to make 40 μm drops. Therefore, cells were only lysed subsequent to drop formation.

FIG. 2c: Microfluidic drop sorting. A forked microfluidic device was used, with one channel for amplification-positive and the other for amplification-negative drops. Dielectrophoresis was used to pull drops into one of the two channels, depending on the fluorescence intensity measured by the PMT.

FIG. 3a: PNA clamping. If the template is wild-type, PNA will remain strongly bound to the DNA, preventing polymerase from amplifying the template. If the template is mutant, polymerase will be able to displace the PNA clamp and amplify the template.

FIG. 3b: PNA clamping. If polymerase is able to displace PNA, it continues across the template and cleaves the Taqman probe, allowing for green fluorescence. Drops with mutant templates appear bright green while those with wild-type templates are pale.

FIG. 4: KRAS Primer synthesis. 12 unique primers were synthesized that would amplify each of the 12 types of KRAS mutation (3 base pair changes possible for the 4 Gaunine nucleotides). Because we had unique primers, the same Taqman probe that was used in the first round of amplification was used in all 12 bar-coded solutions.

FIG. 5: KRT8 Primer testing. Three bright field images (10×) and three fluorescence microscope images (10×) of the drops for testing the KRT8 primer. Green fluorescence indicates positive amplification. The first column shows encapsulated LNCaP cDNA, the second shows PC3 cDNA, and the third shows WBC cDNA. This process was repeated for the 15 other primers.

FIG. 6a and FIG. 6d: The graphs show the distribution of drops based on their duration in milliseconds (corresponding to size) on the x-axis and intensity in volts (corresponding to fluorescence) on the y-axis. Drops that are too small or have merged are therefore not considered, and from the gated drops that concur with size specifications, only those above a certain fluorescence intensity threshold (in this case about 0.2 V) are detected as positive (circled in red). A large majority of drops (98.6% and 97.2%) are gated, indicating minimal loss.

FIG. 6b and FIG. 6e: The histograms depict the distribution of fluorescence intensities for the gated population of drops, with amplification-positive drops to the right of the dotted threshold line. A 10-fold difference can be witnessed from 0.0098% to 0.00092% positive.

FIG. 6c and FIG. 6f: The time plots reveal which specific drops from the number detected are amplification-positive (above the green line). FIGS. 6a-6c are obtained from 50 PC3 and FIGS. 6d-6f are obtained from 5 PC3.

FIG. 7a: Multiplex gel result. The FOLH1, KLK3, and AR bands can all be seen when drops containing LNCaP cDNA and the three primers were broken after dPCR and gel electrophoresis was performed.

FIG. 7b: Negative control. When the sample contained no LNCaP cDNA, and only WBC and RBC cDNA, a negligible number of bright drops were detected, indicating minimal false-positive results.

FIG. 7c: cDNA dPCR dilution experiment. Samples containing cDNA from the equivalent of 50 cells, 5 cells, and 0.5 cells had roughly 10-fold decreases in the number of amplification-positive drops, from 0.0064% to 0.00054% to 0.000039%. Multiple populations of drops are seen below the threshold because of different background signals caused by the various Taqman probes. This does not affect the amplification detection.

FIGS. 8a-8f: Detection of KRAS mutation.

FIG. 8a: For the HT29 cell line (wild-type KRAS), the presence of the PNA clamp inhibited amplification, as the polymerase was unable to displace PNA.

FIG. 8b: For the SW480 cell line (mutant-KRAS), amplification occurred even in the presence of PNA, as seen by the fluorescent drops in both images. Polymerase was able to displace PNA because of mutation in KRAS.

FIG. 8c: Agarose gel result confirming that PNA blocked wild-type amplification; only the second column lacked presence of a 191-bp band.

FIG. 8d: A microfluidic setup could detect as low as one mutant KRAS among 100,000 wild-type genes (0.001% sensitivity). The wild-type control (WT) showed no fluorescent drops, indicating successful clamping by PNA.

FIG. 8e: after PCR, drops with SW480 cells show amplification.

FIG. 8f: after PCR, drops with HT29 cells show no amplification.

FIGS. 9a-9b: KRAS mutation characterization.

FIG. 9a: 12 bar-coded clusters of drops (4 concentrations of Texas Red and 3 concentrations of Alexa 680) were detected. Of these 12, drops from Groups 2 and 7 showed green fluorescence, indicating presence of KRAS mutation.

FIG. 9b: As the drops were bar-coded according to primer used, Group 2 corresponded to the GGT>GTT mutation in codon 12 and Group 7 corresponded to the GGC>GAC mutation in codon 13. Relative mutation frequencies of 55% to 45% are shown in the bar graph, which are consistent with the expected mutation frequencies in SW480 cells.

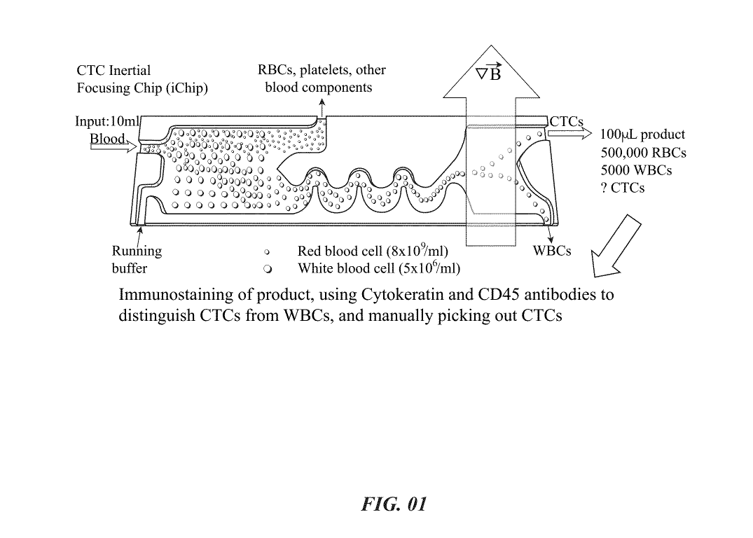

### DETAILED DESCRIPTION

Microfluidics-based technology enables precise control and manipulation of fluids constrained to micron-sized capillaries. Advantages of microfluidics include reduced sample size and reagent consumption, short processing times, enhanced sensitivity, real-time analysis, and automation. More specifically, drop-based microfluidics allows for the creation of micron-sized emulsions that can hold discrete picoliter volumes, with drop-making frequencies of greater than 2,000 drops per second (2 kHz). More recent applications of drop-based microfluidics has led to the development of digital polymerase chain reaction (dPCR), a method that allows for direct amplification and quantification of nucleic acids by generating a multitude of minute reaction vessels (in this case microfluidic drops) in which the conventional PCR can be performed. The drops can hold either individual nucleic acids or a single whole cell (i.e., a complete cell that is not broken or lysed), and thermocycling allows for gene amplification inside the drops. Often, a fluorescence indicator, such as a Taqman probe, is used to depict successful amplification within the drop, and fluorescent drops can be detected or sorted from the others using a flow cytometer. These advantages make microfluidics-based technology most suitable for CTC detection and isolation. For identifying and isolating pure CTCs, a device that combines the resolving power of microfluidics and the amplification power of PCR would be useful. Such a device would achieve the primary goal of identifying and isolating CTCs from the blood, facilitate further understanding of CTC biology, and allow for the development of applications, such as identification of drug resistance phenotypes, that have so far eluded current technologies. In one embodiment, whole genome amplification from a single whole cell is can be performed with a single cell whole genome amplification kit such as GenomePlex® Single Cell Whole Genome Amplification Kit.

The problems associated with sensitive detection of CTCs have also prevented further progress in functional characterization of CTCs. Incomplete information about CTC surface markers seriously limits immunostaining techniques from appropriately differentiating CTCs from other cells in the whole blood. Examining gene expression in cancer cells instead of surface markers may avoid wholly relying the incomplete information about the CTC surface markers. Prostate cancer (PC) is used as an example in this disclosure, as the incidence of PC in the United States is increasing at a rate greater than that of any other cancer, with 238,590 new cases estimated in 2013 alone. By using PC gene-specific primers and fluorophores, we recognized that drop-based dPCR can efficiently determine through an amplification-dependent fluorescence signal whether a nucleic acid or cell expresses PC genes. After reconstituting a whole blood sample to emulate the 100 μL CTC iChip product (about 5×10⁵ RBCs+about 5×10³ WBCs+Arbitrary number of CTCs), encapsulating the sample into drops, and performing dPCR, CTCs are detected and sorted from the rest of the cells, allowing for absolute quantification of CTCs within the blood sample.

KRAS gene mutations in colorectal cancer (CRC) are examined as a test case to demonstrate the versatility and easy adaptability of a microfluidics-based platform in aiding detection and treatment of various cancers. CRC is the second leading cause of cancer mortality in the United States. There are 160,000 new CRC cases diagnosed and 57,000 CRC-related deaths in the United States annually. 30-40% of all CRC cases are associated with mutations within the V-Ki-ras2 Kirsten rat sarcoma viral oncogene homolog (KRAS). The drugs currently available in the market for CRC, including Cetuximab and Panitumumab, target epidermal growth factor receptor (EGFR). An increasing concern about CRC treatment is that patients who have a mutation in the KRAS gene are resistant to EGFR-targeted drug therapy. Due to the acquired resistance to EGFR blockade through KRAS mutation, there is an urgent demand for a test that predicts patient response to EGFR-targeted therapy by determining if there is a mutation in the KRAS gene.

KRAS mutation associated with CRC typically occurs in codons 12 and 13 of the gene, which have the sequence GGT-GGC. A majority of the time, mutations in KRAS occur when one of the Guanine (G) bases have been altered. Thus, there are 12 well-characterized mutations in the KRAS gene. KRAS mutations cluster with twelve possible point mutations in a very short sequence. No method thus far has been able to determine in just one test if the patient has a mutation in KRAS, as current techniques are limited to detecting a single or a small number of point mutations at a time.

Effective targeted treatment for cancer such as using antibodies against epidermal growth factor receptor (EGFR) and antibodies against vascular endothelial growth factor (VEGF) depends on knowledge of genomic characteristics of the cancer cells. For example, therapy using antibody to EGFR greatly benefits from knowledge of specific mutations within the V-Ki-ras2 Kirsten rat sarcoma viral oncogene homolog (KRAS) gene, which are found in 30-40% of colorectal tumors.

Peptide nucleic acid (PNA) is a synthetic non-extendable oligonucleotide that anneals to a complementary strand of DNA and blocks polymerase from binding and replicating the DNA strand. However, even one mismatch between the PNA and the DNA will severely destabilize the PNA-DNA complex and re-enable the binding of polymerase and the process of gene amplification. A PNA clamp that specifically binds to the wild-type KRAS gene and acts as a universal discriminator in the drop-based dPCR system may be used, allowing for any clustered mutation in codons 12 and 13 of the KRAS gene to be amplified and detected through fluorescence signal. dPCR microfluidic technology is best suited to address the problem of low-level gene mutation detection by overcoming limitations of currently used DNA sequencing-based tests.

Methods

1. Cell Culture and mRNA/gDNA Purification

Two PC cell lines, namely LNCaP and PC3, and two CRC cell lines, namely HT29 (with wild-type KRAS) and SW480 (with mutant KRAS), were grown in RPMI medium containing 10% fetal bovine serum and 1% penicillin-streptomycin in a 37° C. incubator. All four adherent cell lines were obtained from American Type Cell Collection and were passaged weekly employing trypsinization. For the PC cell lines, RNA was extracted from cells using Life Technologies RNA-extraction protocol and was reverse transcribed to obtain purified cDNA samples (QIAGEN OneStep RT-PCR Kit). For the CRC cell lines, genomic DNA (gDNA) was extracted using Life Technologies gDNA-extraction protocol.

2. Microfluidic Device Fabrication

Soft lithography techniques were employed to fabricate microfluidic devices. AutoCAD software was used to generate a UV photomask containing micron-sized capillaries of desired structure and dimension. A silicon wafer was coated with UV photoresist, on which the photomask was placed. After UV exposure, the silicon wafer was developed with propylene glycol monomethyl ether acetate (PGMEA) to generate a positive resist with the desired channels exposed. Polydimethylsiloxane (PDMS) was poured atop the positive resist and incubated at 65° C. overnight. After removing the PDMS (now a negative resist with the desired channels) from the silicon wafer, the inlets were punched and the PDMS was bonded to glass via plasma-activated bonding. The devices were treated with hydrophobic Aquapel to prevent the wetting of channels during drop formation. These microfluidic device fabrication methods have been described in detail previously.

3. Preparation of Blood Samples

Whole blood (ZenBio, catalogue #SER-WB10ML) was separated into RBCs and WBCs and reconstituted to contain 500,000 RBCs and 5,000 WBCs to emulate the 100 μL CTC iChip product. PC cells of desired number were then spiked into the mixture, except in control samples.

4. Microfluidic Drop Formation

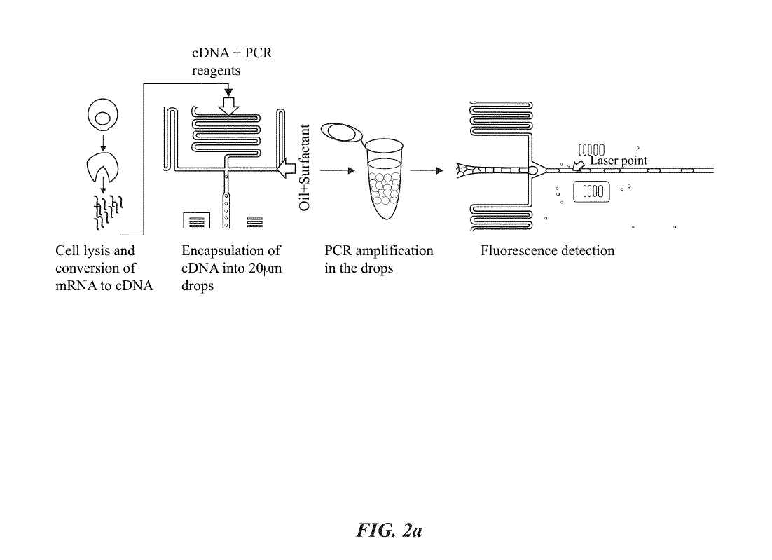

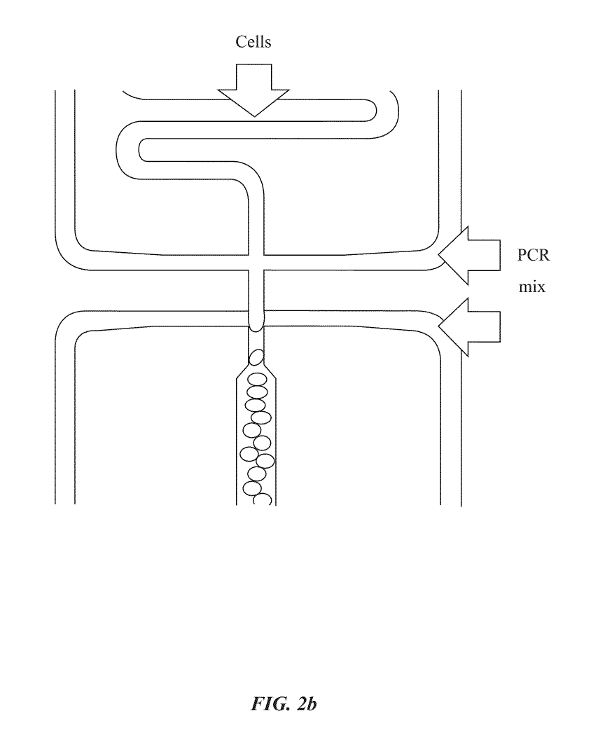

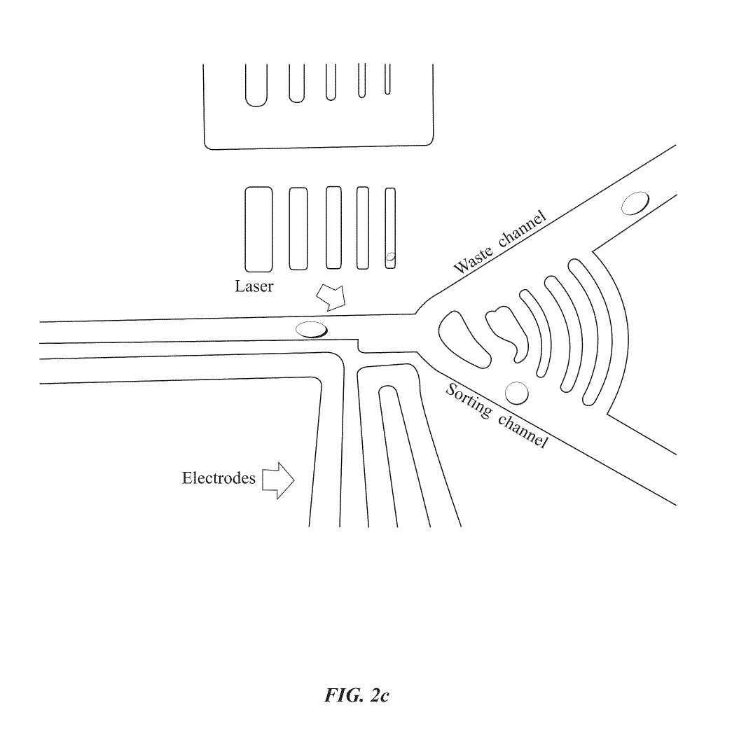

Two drop-makers were employed: a two-inlet 20 μm drop-maker for the cDNA dPCR and KRAS mutation detection, and a three-inlet 40 μm drop-maker for the single-cell dPCR. For the 20 μm drop-maker, HFE-7500 fluorinated oil with 1.5% fluoro-surfactant was inserted into one inlet while the cDNA (or gDNA in the case of KRAS mutation detection) sample mixed with the PCR reagents was inserted into the other inlet (FIG. 2a). For the 40 μm drop-maker, HFE-7500 fluorinated oil with 1.5% fluoro-surfactant was inserted to one inlet, the cell sample was inserted into the second inlet, and the PCR reagents containing lysis buffer were inserted into the third inlet (FIG. 2b). In this case, the PCR mixture and lysis buffer were co-encapsulated with the cell sample in the drop-making device. A vacuum was applied at the outlet to generate drops at about 2 kHZ following techniques described previously.

5. Digital PCR

The PCR mixture included 5× concentrated buffer, dNTP, enzyme polymerase, forward and reverse primer, Taqman probe, RNase Inhibitor, BSA, 10% Tween20, 25% NP40, and the cDNAs from LNCaP, PC3, and WBCs. For KRAS mutation detection, gDNAs from HT29 and SW480 as well as the PNA clamp were added to the PCR reagents instead of cDNA. After encapsulation, thermocycling of the drops was performed with an initial denaturation step at 95° C. for 10 minutes; followed by 40 cycles of: 95° C. for 30 seconds, 70° C. for 10 seconds, 53° C. for 30 seconds, and 62° C. for 50 seconds; and lastly 62° C. for 10 minutes. For single-cell dPCR, 10× lysis buffer (Cell Signaling) was used in place of NP40 in the PCR mixture, and no cDNA was added to the samples. To perform single-cell encapsulation, cell samples were put in a drop-maker that allows for the cells and the PCR mixture to be co-encapsulated into 40 μm drops. After encapsulation of the single cells, a 40-minute 50° C. reverse transcription (RT) step was performed, followed by the aforementioned thermocycling procedure.

6. PNA Clamping for KRAS Mutation Detection

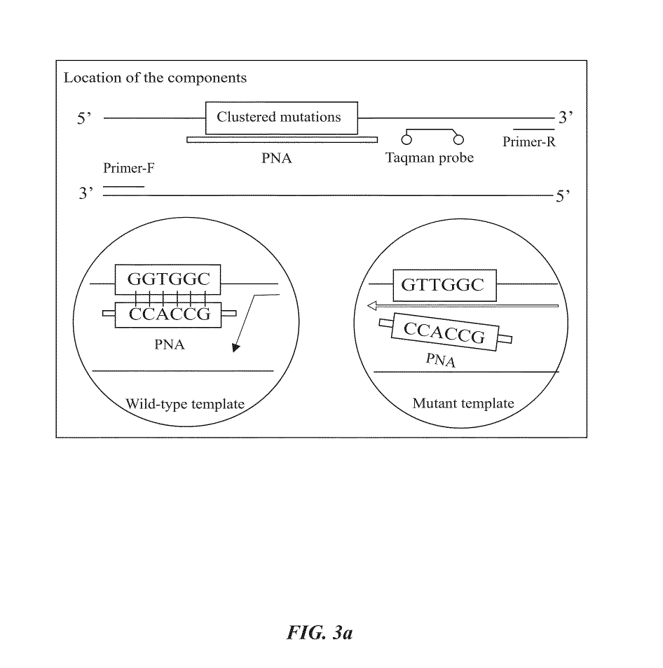

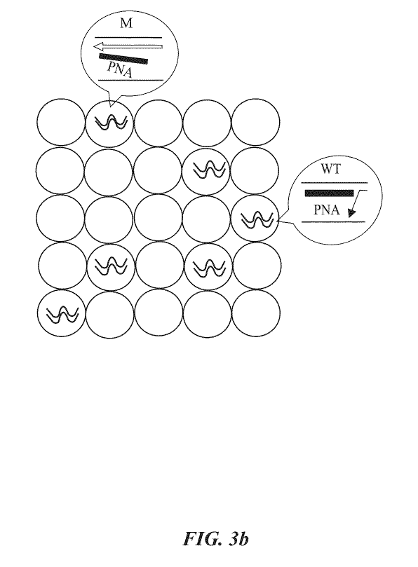

A 17-bp PNA clamp was synthesized complimentary to the wild-type KRAS sequence. In presence of a mutation, polymerase was able to displace the destabilized PNA molecule and elongate the strand. Downstream of codons 12 and 13 of the KRAS gene, where the PNA would bind if the template were wild-type, was a fluorescin amitide-minor groove binder (FAM-MGB) Taqman probe containing a FAM fluorophore and MGB quencher molecule. When the polymerase was able to displace the PNA molecule in the case where there was a mutation, the polymerase would also cleave the Taqman probe, liberating the fluorophore from the quencher and allowing for bright green fluorescence (FIG. 3a). In contrast, the drops containing wild-type templates in which PNA had strongly clamped the DNA did not fluoresce and remained pale green due to blocked amplification (FIG. 3b). Subsequently, the drops containing mutant KRAS sequences may be identified and separated; and the content of these drops containing mutant KRAS sequences may be further amplified using a suitable method. This two-step amplification method enables detection of a mutant KRAS sequence in the presence to more than 100,000 copies of wild-type KRAS sequence.

7. Drop Detection and Cell Sorting

Fluorescence microscopy was used to image drops after dPCR. Quantitative detection of bright drops was performed with a microfluidic chip-based flow cytometer system (FIG. 2a).

As drops flowed past a laser spot at a high frequency of approximately 500 Hz, fluorescence measurements from each drop were collected through the objective and analyzed by a photomultiplier tube, or PMT. The duration of a drop passing the laser gave indication of the drop size. In this case, the PMT had a wavelength of 488 nm (excitation peak for FAM).

LabVIEW software was employed for drop detection data analysis. For cell sorting, a forked microfluidic device was fabricated, with one channel for amplification-positive and the other for amplification-negative drops. Employing the same PMT setup, dielectrophoresis was used to pull drops into one of the two channels, depending on the fluorescence signal (FIG. 2c). Microfluidic drop-based detection and sorting have been detailed previously.

8. KRAS Mutation Characterization

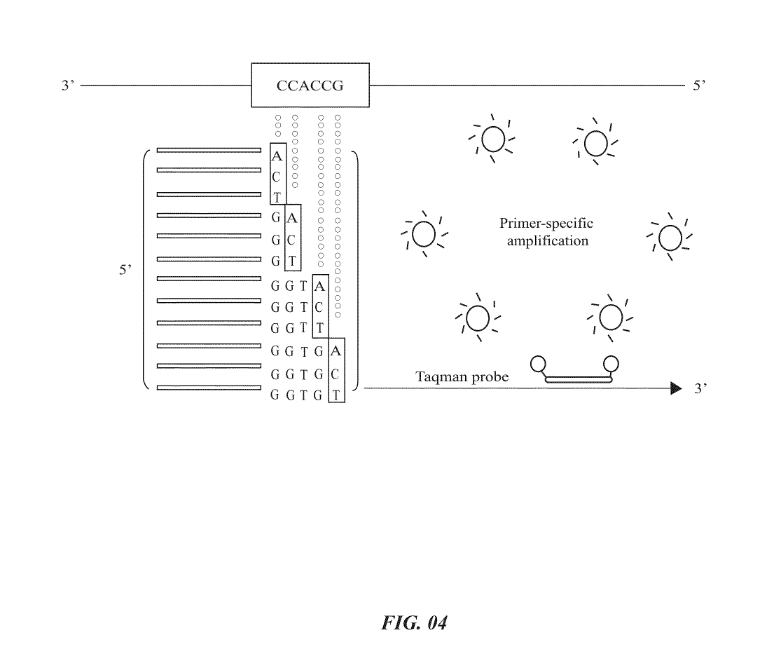

After sorting out amplification-positive drops (all which contained mutant templates, as wild-type templates were clamped by PNA), these drops were broken using Perfluorooctanoic acid (PFO) and diluted with water to achieve an average of 1 amplicon per 10 drops for the second round of encapsulation. To characterize the twelve types of single-nucleotide KRAS mutations in codons 12 and 13 in just one experiment, 12 corresponding primers were designed (FIG. 4). The diluted sample was split into 12 tubes, and each was mixed with a unique PCR solution containing one of the 12 designed primers. The 12 solutions were fluorescence bar-coded by using 12 different combinations of Texas Red and Alexa 680 dyes (4 concentrations of Texas Red and 3 concentrations of Alexa 680). The 12 solutions were then encapsulated simultaneously through 12 parallel microfluidic drop-making devices. After dPCR was performed, drops were detected with three PMT's: one for FAM at 488 nm, one for Texas Red at 615 nm, and one for Alexa 680.

9. Confirmation of Amplicon

During initial rounds of primer testing and cDNA dPCR, drops were broken using Perfluorooctanoic acid (PFO), and gel electrophoresis was completed to ensure that the amplicon was of expected length. 1% agarose gels were imaged using UV excitation.

Results

cDNA Digital PCR

Two common PC cell lines, PC3 and LNCaP, were used to mimic prostate CTCs. 16 specific primers and their respective Taqman probes were obtained. Through prior deep sequencing experiments, these primers have been shown to amplify PC genes, which hybridize with their respective Taqman probes. The first step was to determine which of the 16 predetermined primers could be used to properly amplify PC-specific genes and emit green fluorescence signal within the drops. In addition to the LNCaP and PC3 PC cell lines, WBCs were used as a negative control to ensure that these primers did not amplify any WBC genes. As each cell contains only two copies of each gene in its genome, it was determined that direct PCR amplification would result in a very low fluorescence signal. Since each cell releases several hundreds of mRNA molecules per gene into the cytoplasm, performing RT would provide cDNA copies in manifold concentration to obtain a better signal within the drops. Each of the two cell lines and the WBCs were therefore lysed, their mRNA was extracted, and bulk RT was performed to convert mRNA into cDNA, as shown in FIG. 2a. Each cDNA sample was diluted such that it would have a Poisson distribution parameter of 0.1, meaning that one in every ten drops would contain a cDNA molecule. After encapsulation and cDNA dPCR, the drops were examined under a fluorescence microscope to determine which primers amplify PC-specific genes and show signal in the three cell types (FIG. 5). The 16 primers were divided into 5 categories to cover all possible genetic expression of PC cells, and each primer gave a positive or negative result for the amplification of the cancer-specific genes (Table 1). To confirm whether drops truly contained the genes of interest, they were broken and gel electrophoresis was performed with the PCR product. Gel results corroborated with those from cDNA dPCR.

TABLE 1
Primer testing. 16 primers from 5 different categories (prostate, mesenchymal,
proliferation, epithelial, and stem cell) were tested using cDNA dPCR for each
of the three cell types. Amplification was confirmed with gel electrophoresis.

| | 1. Prostate | | | | | | | Control |
|---|---|---|---|---|---|---|---|---|
| Primers | AR | KLK3 | FOLH1 | AMACR | KRT8 | KRT18 | KRT19 | GAPDH |
| LNCaP | + | + | + | + | + | + | + | + |
| PC3 | − | − | − | − | + | + | + | + |
| WBC | − | − | − | − | − | − | − | + |

| | 2. Mesenchymal | | 3. Proliferation | | 4. Epithelial | | 5. Stem Cell | |
|---|---|---|---|---|---|---|---|---|
| Primers | FN1 | Serpine1 | MK167 | CCND1 | EpCAM | KRT7 | SOX4 | Nanog |
| LNCaP | − | − | − | − | − | + | − | − |
| PC3 | + | + | + | + | + | + | + | + |
| WBC | − | + | − | − | − | − | − | − |

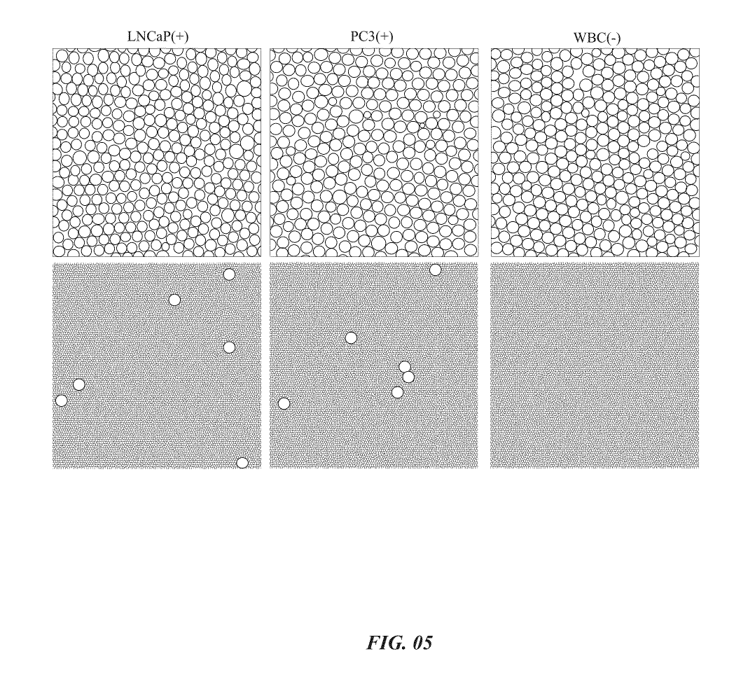

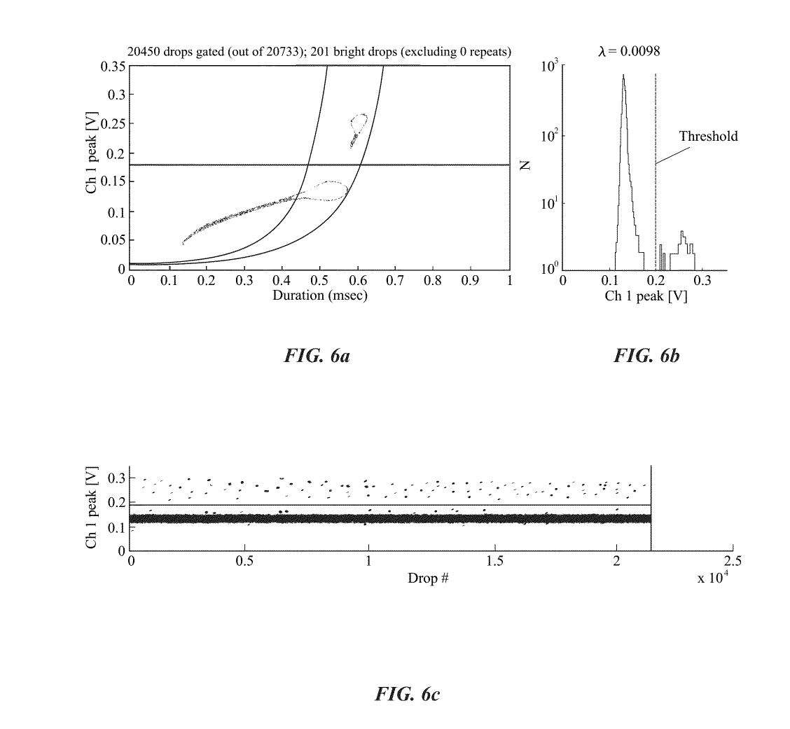

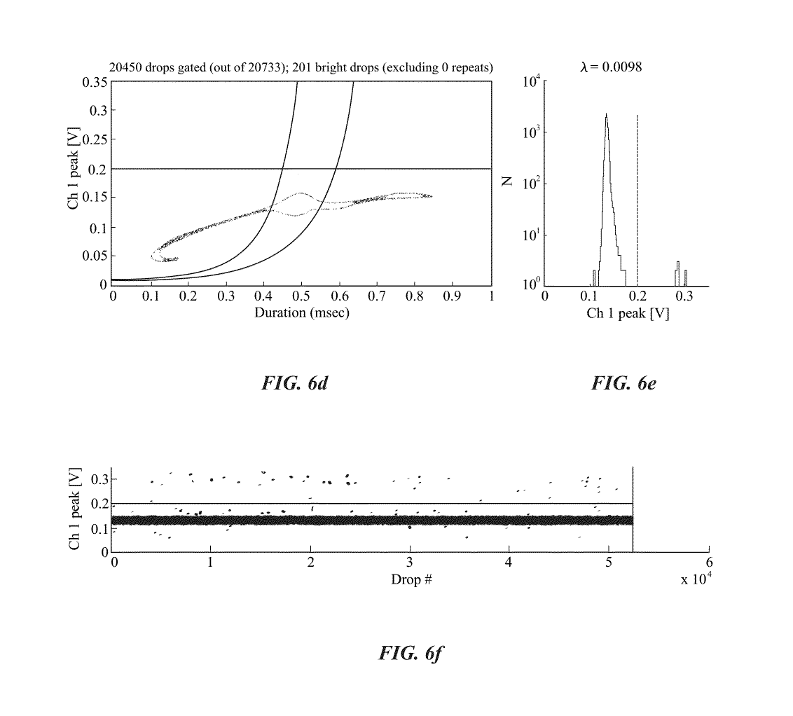

To more accurately emulate the 100 μL iChip product, the prostate cell lines were spiked into a 100 μL blood sample containing 500,000 RBCs and 5,000 WBCs. After lysing the cells to extract mRNA and performing RT to convert to cDNA, the cDNA samples were encapsulated and dPCR was performed for the amplification of PC-specific genes, resulting in fluorescence of amplification-positive drops. Subsequently, drops were quantitatively detected for fluorescence using a microfluidic chip-based flow cytometer system. To test the accuracy of the dPCR and detection mechanisms, the well-known EpCAM primer was used with a sample containing 50 PC3 cells and a second sample containing 5 PC3 cells. An approximately 10-fold decrease in the number of bright drops was seen between the two samples, as the amplification-positive detection rate decreased from 0.0098% to 0.00092%, about 10 fold reduction (FIG. 6a-f). This control experiment, with a decrease in detection rate consistent with the decrease in input cDNA concentration, confirms the robustness and reproducibility of the dPCR as well as the detection.

Due to the high heterogeneity of CTCs, employing multiple primers and performing “multiplex” amplification would detect as many CTCs as possible. After completing analysis of both the fluorescence images after dPCR and the gel results, the AR, KLK3, FOLH1, AMACR, KRT8, KRT18, and KRT19 primers were found to be most promising in successful and reproducible amplification in the LNCaP and PC3 cell lines, but not in the WBCs, which need to be differentiated from the PC cell lines that mimic the CTCs from the true sample. AR, KLK3, FOLH1, and AMACR are able to detect LNCaP cells while KRT8, KRT18, and KRT19 are able to detect both LNCaP and PC3 cells. These seven prostate primers were chosen over other primers (being mesenchymal, proliferation, epithelial, and stem cell), which also detected the prostate cell lines, because using only PC-specific primers would ensure fewer false-negative results and allow for unequivocal discrimination of PC cells from the rest. However, as each primer pair requires its own Taqman probe that can cause low levels of fluorescence even without amplification, it is important that the multiple primers used do not present a background signal that makes it difficult to distinguish amplification.

When all seven primers and Taqman probes were used, accurate detection was not possible. A mix of 100 LNCaP and PC3 cells was spiked into 500,000 RBCs and 5,000 WBCs, and after lysis and RT, cDNA dPCR was performed with all seven primers and their respective Taqman probes. However, because of the increased background, two distinct clusters of fluorescence intensity were not observed.

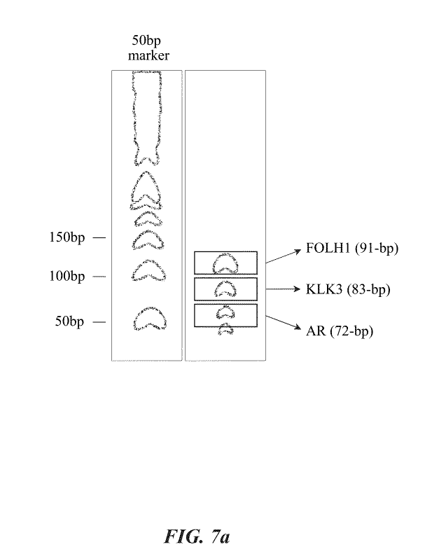

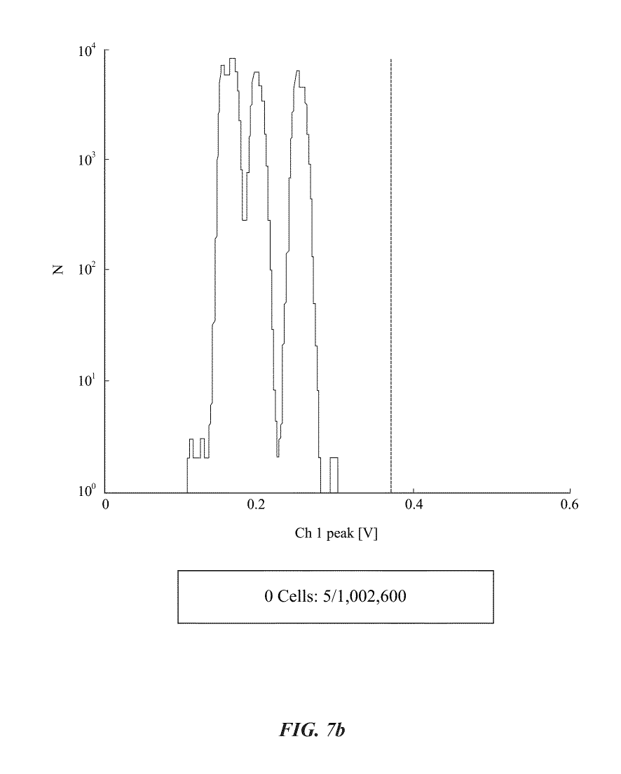

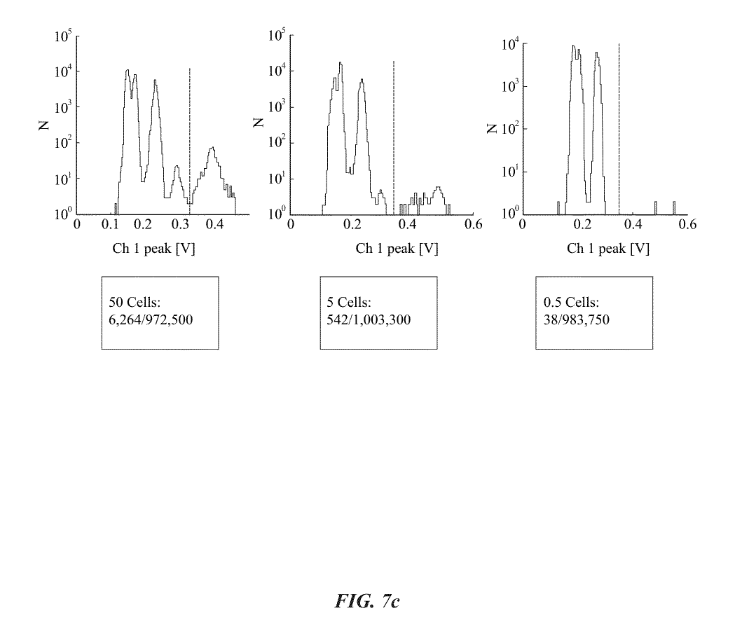

Optimization experiments suggested that only AR, KLK3, and FOLH1 primers could be used for maximal signal and minimal background. The results showed successful multiplex amplification using these primers (FIG. 7a). The negative control sample, containing no prostate cDNA whatsoever, showed minimal bright drops (FIG. 7b). This result is essential as it demonstrates there is no false-positive signal during multiplexing when only cDNA molecules of RBCs and WBCs are present. Three distinct samples containing LNCaP cDNA equivalent to 50 cells, 5 cells, and 0.5 cells were spiked into 500,000 RBCs and 5,000 WBCs. There were ten-fold decreases in number of bright drops detected between the three samples (FIG. 7c).

Single-Cell Digital PCR

Encapsulating cDNA after lysing cell samples, performing dPCR, and detecting for fluorescence is a promising approach for the early detection of CTCs in the blood sample. The method allows for absolute quantification of CTC transcripts obtained from a liquid biopsy in just a few hours. However, a limitation of this strategy is that after detection, genetic information about a single CTC cannot be obtained, as the cells were initially pooled together and lysed before the encapsulation step. If an intact CTC could be individually encapsulated, followed by lysis and RT-PCR within each drop, bright drops could be sorted out and the genetic information from a single cell could be retrieved from an individual reaction vessel. Further, the single-cell approach would allow the number of cells to be directly quantified, without relying on cDNA as a surrogate. This method has been described, although never before practiced for CTCs. The workflow for single-cell dPCR is described in FIG. 2b.

A dilution experiment was conducted in which 50, 20, and 5 PC cells (with LNCaP to PC3 ratio of 1:1) were spiked into three samples of 500,000 RBCs and 5,000 WBCs, encapsulated, and single-cell dPCR was performed. 50, 20, and 5 PC cells were obtained through serial dilutions of the initial cell solution. In this case, all seven prostate primers (AR, KLK3, FOLH1, AMACR, KRT8, KRT18, and KRT19) were multiplexed, and it can be seen in Table 2 that comparable numbers of cells as spiked in the samples were detected as positive.

TABLE 2
Single-cell dPCR dilution experiment. Roughly all cells that were
present in the sample were detected in each case. As varying numbers
of prostate cells were spiked into samples by dilution and not
by exact quantification, obtaining precisely the correct number
of bright drops was not expected. For the negative control with
no PC cells, no bright drops were detected.

| | 50 PC Cells | 20 PC Cells | 5 PC Cells | 0 PC Cells |
|---|---|---|---|---|
| Positive Drops | 38 | 15 | 4 | 0 |
| Capture Efficiency | 76% | 75% | 80% | N/A |
| Total drops | 711,100 | 778,160 | 947,810 | 669,280 |

Using all seven primers in the case of single-cell dPCR does not lead to unnecessary background as it does in the cDNA dPCR, because amplification-positive drops now have significantly more starting material (not just one cDNA molecule) to differentiate between a drop containing a CTC and a drop with just background signal. The bright drops were then sorted from the rest in a microfluidic device using dielectrophoresis to obtain a pure CTC sample. Results showed that the drop-based single-cell dPCR method can be successfully used to detect CTCs that are in extremely low concentration. The multiplexing of seven PC gene-specific primers allows for a heterogeneous population of PC cells to be detected.

KRAS Mutation Detection

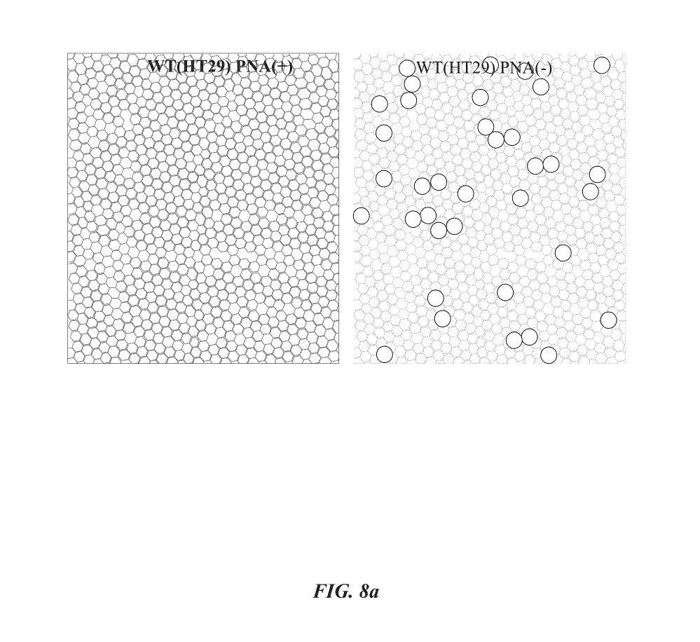

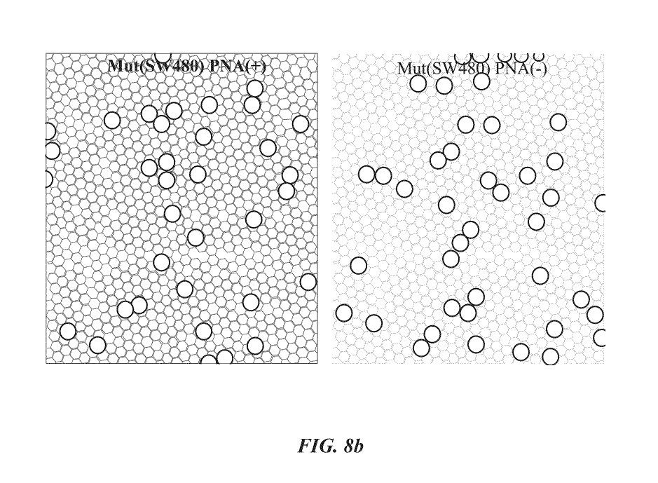

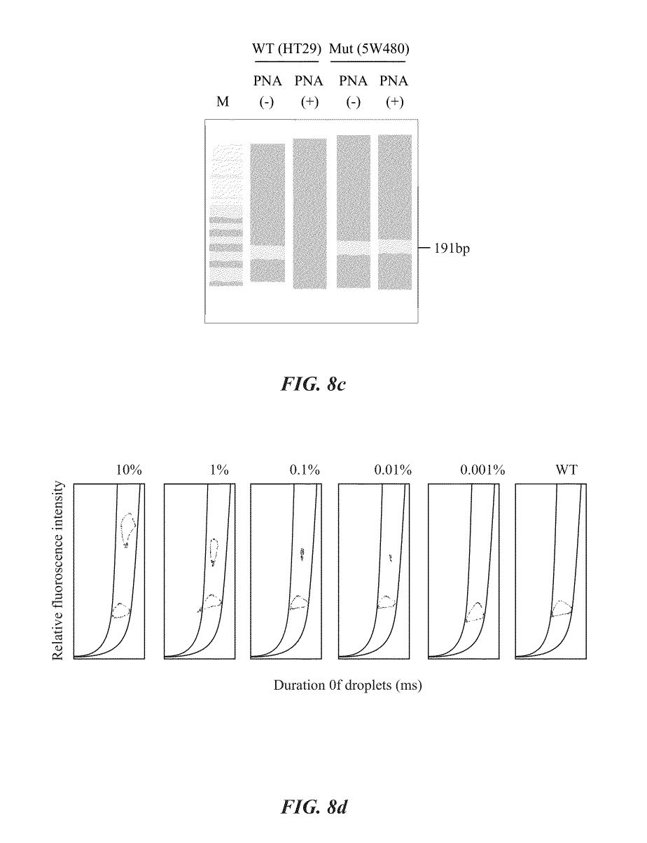

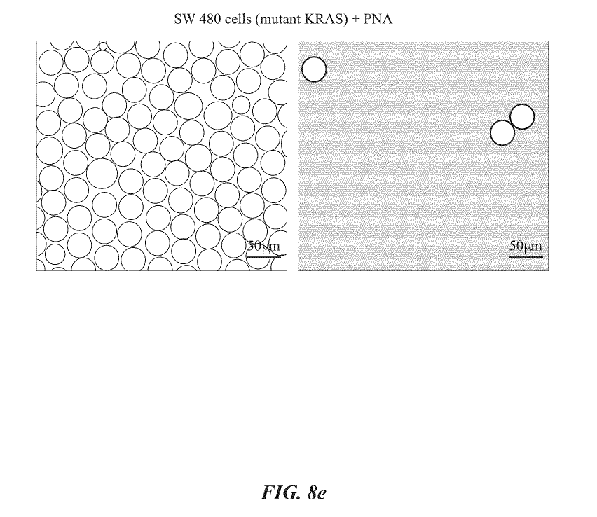

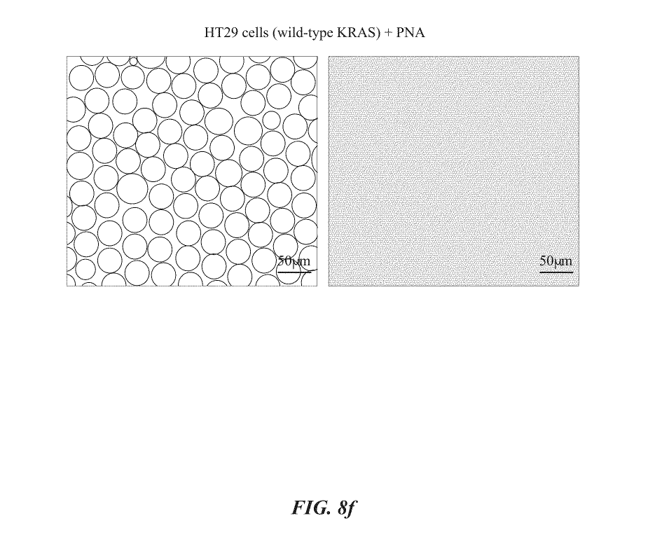

The efficiency of the PNA clamping was tested by encapsulating and amplifying wild-type HT29 gDNA in drops. As expected, in the absence of PNA, bright drops were seen due to the cleavage of the Taqman probe by polymerase. The percent of bright drops was between the range of 0.09 and 0.11, consistent with the Poisson parameter of 0.1. Further, when PNA was added to the PCR mixture, no bright drops were seen, as its presence blocked the polymerase from completing amplification and separating the fluorophore from the quencher (FIG. 8a). To investigate whether a mutation in KRAS destabilized the PNA molecule enough to allow for amplification, mutant SW480 gDNA were encapsulated into drops with and without PNA. SW480 cells harbor either a GTT mutant at codon 12 or a GAC mutant at codon 13. As shown in FIG. 8b, the amplification of the mutant sample was not affected by presence of PNA, and the same ratio of bright drops was observed in both cases. Thus, it was confirmed that there was no PNA clamping effect on DNA sequences that have even one mutation site. FIG. 8c depicts an agarose gel electrophoresis result that further indicates that PNA clamping effectively occured only for wild-type templates. Amplification bands of the expected size (191-bp) were seen in all cases where PNA was absent or where mutant gDNA had been used.

To test for the sensitivity of this assay, a dilution experiment was performed. The SW480 gDNA was serially diluted in HT29 gDNA by 10-fold, down to one mutant KRAS template in 100,000 wild-type templates. Drops containing the mutant KRAS template generated a relative fluorescence intensity of 0.6, compared to the signal of 0.3 present in the drops containing the wild-type templates. A threshold of 0.55 was used to assign each drop as a positive or negative. As shown in FIG. 8d, the number of bright drops varied accordingly with the initial concentration of mutant templates, indicating that the system performs within a wide range.

The efficiency of the PNA clamping was also demonstrated by co-encapsulating whole cancer cells (e.g., colorectal cancer cells), a PCR mixture, lysis buffer and PNA. The lysis buffer lyses the whole cells during encapsulation. PCR reaction can be carried out in each drop with a now-lysed cell but the DNA templates in the drop originated from only that cell. In one example, as shown in FIG. 8e and FIG. 8f, a mixture of HT29 cells (with wild-type KRAS gene) and SW480 cells (with mutant KRAS gene) is subject to this process. Drops with HT29 cells encapsulated therein do show fluorescent signal, which indicates that the wild-type KRAS gene sequence is completely clamped by the PNA. Drops with SW480 cells show fluorescent signal.

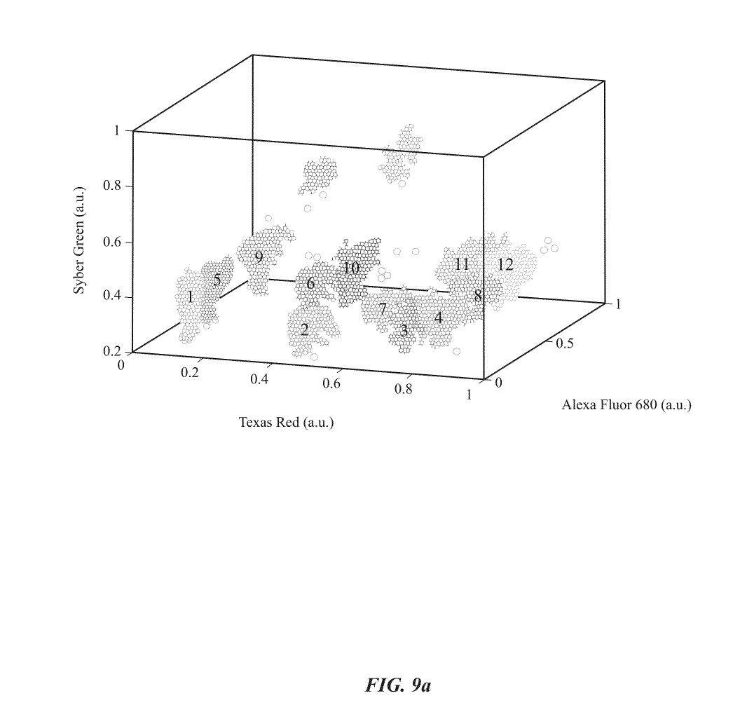

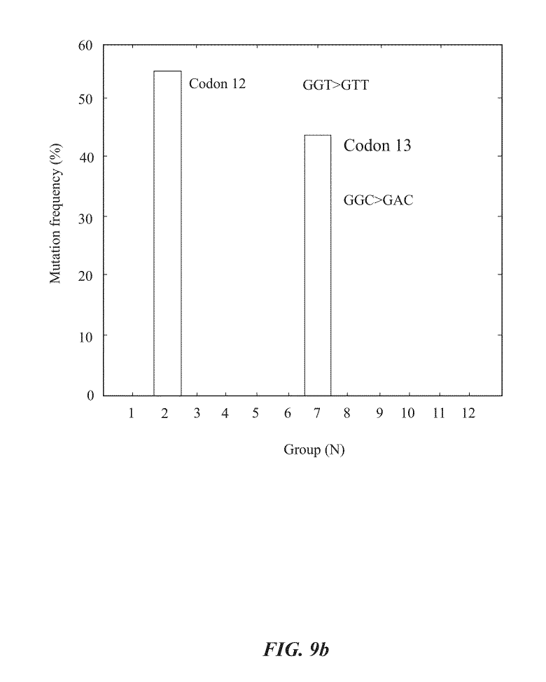

Many studies have indicated that the development, prognosis, and treatment of CRC are related to the specific KRAS mutation patterns that exist in the patient. Thus, the precise characterization of KRAS mutations, in addition to the determination of the presence and rate of mutation, would throw light on the exploration of the clinical significance of unique mutations. FIG. 9a shows a three-dimensional plot with 12 different bar-coded clusters. A significant portion of Groups 2 and 7 are above the rest of the clusters in the vertical dimension, indicating presence of green fluorescence and therefore positive amplification. Group 2 corresponds to the primer that amplifies the mutation GTT-GGC (replaced G with T in codon 12), and Group 7 corresponds to the primer that amplifies the mutation GGT-GAC (replaced G with A in codon 13). FIG. 9b shows that the percentage of each mutation can be easily quantified. In the experiment present, the relative frequencies of Group 2 and Group 7 mutations were 55% and 45%, consistent with the expected results from the SW480 cell line.

Discussion

The platform for single-cell dPCR screening is successful in detecting and isolating pure CTCs, as discussed below. Previous reports of CTC isolation methodologies relied on physical properties such as size, or few known cell surface markers in combination with microscopic techniques. Although these studies resulted in incremental advances in CTC isolation, heterogeneity of CTCs coupled with lack of well-defined cell surface markers implied that any one of these techniques is inadequate for detection and isolation of CTCs from blood samples. This problem can be addressed by combining dPCR with a microfluidics system to identify CTCs based on gene expression. In one method, individual cDNA molecules are encapsulated, and in another, intact single CTCs are encapsulated. Both methods allow for the diagnosis of low-level CTCs from a blood sample. Similar tests were also successfully completed by the mentor on blood samples from PC patients.

The results from Table 2 are highly significant and point to many important advances made by the screening platform. The negative control sample, containing no PC cells whatsoever, showed no bright drops. This result is essential as it demonstrates that there is no false-positive signal during multiplexing when only cDNA molecules of RBC and WBC are present. Next, the capture efficiency ranged from 75-80%, which is quite high. Finally, as the number of bright drops is less than the number of PC cells introduced, it is reasonable to conclude that no PC cells were fragmented prior to encapsulation. Since the total number of drops exceeded the total number of RBCs, WBCs and PC cells in the blood sample and the cells in the blood sample were randomly distributed, it could be inferred that statistically each drop contains only one cell at maximum. This suggests that no cell escapes sampling, and that by sorting out amplification-positive drops from the rest, a pure CTC sample has been obtained.

By performing single-cell dPCR, the number of bright drops is less than or equal to the number of CTCs. On the other hand, as there are multiple cDNA molecules per CTC, many more bright drops than the number of CTCs are seen in cDNA dPCR after RT and individual encapsulation of each cDNA molecule. However, in the single-cell experiments, the fact that each drop contains a single cell, which is subsequently lysed and subject to reverse transcription, individual cDNA molecules are not isolated from one another, and the fluorescence signal from a single drop after single-cell dPCR is much stronger than that from a single drop after cDNA dPCR. The stronger signal and downstream applications from using single-cell dPCR are major advantages of this method. The dPCR platform is the first that addresses the problems of tumor heterogeneity and CTC rarity by using multiple primers and compartmentalizing amplification reactions. By isolating a pure sample of CTCs from the bloodstream, these cells can be characterized and their genomes can be sequenced, shedding light upon the patient's cancer.

The microfluidics platform has also shown one example of cancer cell characterization by detecting rare KRAS mutations from CRC cells. Currently, detection of mutations in KRAS genes is done by traditional Sanger DNA sequencing methods that can only detect mutations in the KRAS gene when the allele frequency of the gene mutation is between 10-20%. Next-generation deep sequencing methods do improve detection thresholds to 1%, but KRAS mutations implicated in CRC have even lower frequencies. Quantitative real-time polymerase chain reaction (qPCR), which also has a detection threshold of 1%, cannot detect KRAS mutations from cancer samples, as background signal from non-specific templates overwhelm KRAS-targeted amplification. None of these methods suffices for adequate exploration of highly heterogeneous cancer samples, which require thresholds of 0.1% or even lower. The platform, however, can reliably detect as little as one copy of mutant KRAS template in the presence of 100,000 wild-type templates. Through compartmentalization, the dPCR technique decreases noise and greatly increases the signal-to-noise ratio of low-level targets. This technique provides for an unprecedented sensitivity; it is at least 10,000 times more sensitive than Sanger sequencing and 1,000 times more sensitive than qPCR and deep-sequencing techniques, which are also far more expensive. This highly specific and sensitive mutation detection system is capable of accurately and absolutely quantifying mutant templates within a sample. Thus, sensitivity is only limited by the number of molecules that can be analyzed in a given time period. The microfluidic technique further characterizes which KRAS mutation the patient has through a novel bar-coded microfluidic drop-based method. Traditional methods of detecting rare mutations involve extensive sequencing of cloned products or expensive and complicated deep sequencing methods. However, even these techniques cannot characterize mutations that occur below a certain threshold. This novel microfluidic technique overcomes the challenge of detecting and characterizing low-abundance mutations.

The isolation of pure circulating tumor cells followed by PNA clamping-based quantitative detection and rapid characterization of clustered mutations as presented would significantly benefit both cancer diagnosis and therapy.

Thomas Ashworth, the first scientist to observe CTCs in 1869, postulated “cells identical with those of the cancer itself being seen in the blood may tend to throw some light upon the mode of origin of multiple tumours existing in the same person”. This disclosure describes a breakthrough microfluidic technique known as drop-based dPCR for the quantitative detection of rare CTC genes and CTCs from blood samples. This technique can both detect and isolate a single CTC from the blood in a single drop. The CTC detection system is very flexible. In future, as more primers are determined for amplification of prostate cancer genes, they can be implemented into the dPCR technique to increase the scope of prostate cancer cell detection. Further, the platform can easily be adapted for the detection of CTCs from a broad range of cancers. Importantly, sample enriching steps similar to those described in the CTC iChip can be built upstream of the device, thus allowing for automation of the process.

As each drop statistically only contained one cell, and only drops that contained CTCs gave amplification-dependent signal, it can be inferred that it is possible to obtain a 100% pure CTC product from CTCs that were originally among billions of other cells in the blood sample. Since fluorescent drops that contain individual CTCs were sorted from the rest of the population, these isolated CTCs can now be characterized individually. By preserving a complete CTC genome in each drop, sequencing results could give new insight on patients' cancer progression and allow for individualized, targeted drug therapy depending on the specific mutations found in the patient's CTCs. This microfluidics approach would revolutionize cancer biology by informing which underlying mutations in the CTCs are responsible for the cause and spread of cancer.

The study also allows absolute quantification of low-abundant KRAS mutations through PNA clamp-facilitated drop-based digital PCR and accurate determination of KRAS mutation rates. Previous work of CTC isolation has correlated the number of CTCs with the clinical course of disease, but has not provided detailed analysis of the genetic mutations in CTCs due to the limited resolution of the previous techniques such as fluorescence in situ hybridization (FISH) or immunostaining. The study represents a major advancement by adopting techniques such as PNA clamping to mask wild type loci and selectively amplify mutant genetic loci, thus identifying CRC drug sensitivity. Exact characterization of KRAS mutations at the single-molecule level can be used in the stool, blood, or other patient sample and provide a potentially noninvasive means for predicting the efficacy of EGFR-targeted therapy in CRC patients. In future, by characterizing KRAS mutations, doctors can administer individualized therapy based upon the specific mutation patterns of the patient and better predict the prognosis of the disease. The techniques disclosed herein can be used for any clustered mutation, so long as gene-specific primers, a complementary PNA clamp, and a proper Taqman probe are synthesized for the dPCR reaction.

A combination of the CTC detection and isolation platform with a cancer cell characterization technique similar to the KRAS mutation detection platform would allow for early cancer detection and treatment. The disclosure may be extended to isolating and detecting CTCs for breast and lung cancers, and may include a universal microfluidic platform for the early diagnosis and treatment of cancer.

What is claimed is:

1. A method for diagnosing cancer in a person or animal, comprising:
Obtaining or preparing a sample comprising cDNAs of a plurality of genes of the person or animal;
encapsulating the cDNAs into discrete droplets, wherein statistically each of the discrete droplets contains at most one of the cDNAs;
amplifying the cDNAs in the droplets; and
determining whether the droplets contain a cDNA of a mutation of a V-Ki-ras2 Kirsten rat sarcoma viral oncogene homolog (KRAS) gene.

2. The method of claim 1, further comprising sorting the droplets.

3. The method of claim 1, wherein the sample is a whole blood sample.

4. The method of claim 1, wherein obtaining the sample comprises reverse transcribing mRNAs.

5. The method of claim 1, wherein the cancer is colorectal cancer.

6. The method of claim 1, wherein the cancer is prostate cancer.

7. The method of claim 1, wherein the mutation is codon 12 or codon 13 of the KRAS gene.

8. The method of claim 1, wherein the mutation is alteration of a guanine in the KRAS gene.

9. The method of claim 1, wherein determining whether the droplets contain a cDNA of a mutation of the KRAS gene is by using peptide nucleic acid (PNA) clamping.

10. The method of claim 1, wherein determining whether the droplets contain a cDNA of a mutation of the KRAS gene is by using a fluorescence indicator.

11. A method for diagnosing cancer in a person or animal, comprising:
obtaining or preparing a sample comprising whole cells of the person or animal;
encapsulating the whole cells into discrete droplets, wherein statistically each of the discrete droplets contains at most one of the whole cell;
lysing the whole cells in the droplets;
forming cDNAs by reverse transcribing mRNAs in lysate in the droplets;
amplifying cDNAs in the droplets; and
determining whether the droplets contain a cDNA of a mutation of a KRAS gene.

12. The method of claim 1, further comprising sorting the droplets.

13. The method of claim 11, wherein the sample is a whole blood sample.

14. The method of claim 11, wherein the cancer is colorectal cancer.

15. The method of claim 11, wherein the cancer is prostate cancer.

16. The method of claim 11, wherein the mutation is codon 12 or codon 13 of the KRAS gene.

17. The method of claim 11, wherein the mutation is alteration of a guanine in the KRAS gene.

18. The method of claim 11, wherein determining whether the droplets contain a cDNA of a mutation of the KRAS gene is by using peptide nucleic acid (PNA) clamping.

19. The method of claim 11, wherein determining whether the droplets contain a cDNA of a mutation of the KRAS gene is by using a fluorescence indicator.

20. The method of claim 1, wherein the person is suspected of having cancer.

21. The method of claim 11, wherein the person is suspected of having cancer.

22. The method of claim 1, further comprising determining the sequence of the mutation.

23. The method of claim 11, further comprising determining the sequence of the mutation.

24. The method of claim 22, further comprising selecting a therapy for the person based on the sequence of the mutation.

25. The method of claim 23, further comprising selecting a therapy for the person based on the sequence of the mutation.

26. The method of claim 24, wherein the therapy comprising introducing an antibody into the person.

27. The method of claim 25, wherein the therapy comprising introducing an antibody into the person.
